(19) United States
(12) Patent Application Publication
(10) Pub. No.: US 2006/0106356 A1
(43) Pub. Date: May 18, 2006

(54) Urine-absorbing pad

(76) Inventors: Caren S. McVicker, Wayland, MA (US); Christine S. Fry, Brandon, MS (US)

Correspondence Address: ABELMAN, FRAYNE & SCHWAB, 150 East 42nd Street, New York, NY, 10017-5612, US

(21) Appl. No.: 10/992,264

(22) Filed: Nov. 18, 2004

(51) Int. Cl. A61F 13/15

(52) U.S. Cl. 604/385.01; 604/385.101

(57) ABSTRACT

A urine-absorbing pad assembly for use without disrobing and while the wearer is standing includes an absorbent member for receiving and retaining urine and a supporting member for the absorbing member that assists in manually positioning the absorbing member in the wearer's groin between the wearer's clothing and body in preparation for use. The supporting member is optionally separable from the absorbent member to serve as a reusable applicator to assist in positioning the pad, after which it can be withdrawn, either before use or following removal from the wearer and prior to disposal of the pad.

### FIELD OF THE INVENTION

The present invention relates to absorbent pads and specifically to urine-absorbing pads.

### BACKGROUND OF THE INVENTION

Absorbent pads of the prior art have a broad range of applications, and are configured and constructed for being worn for extended periods of time to contain the exudates of a child or an adult. Absorbent pads typically include a liquid permeable layer adjacent to the skin of the wearer, one or more absorbing layers, and an exterior liquid impermeable backing. Absorbent pads often have fasteners, such as adhesive portions, to keep them in position while they are worn. Many absorbent pads also include one or more biasing members for retaining the absorbent pad in an optimal position in the groin of the wearer and assisting in providing a seal along the perimeter of the absorbent pad.

It is known in the prior art to provide an absorbent pad with reinforcement that is elongated and stiffened in a transverse direction so that it can temporarily deform or twist to comfortably accommodate bodily movements of a wearer and then return to its original shape. These prior art reinforcing structures are configured to preclude the bunching and twisting of the absorbent pad transversely or between the legs of the wearer during extended use. Reinforcing structures achieve their stiff and resilient characteristics by their selection of materials and structural shapes. Materials for reinforcing structures include spring wire, plastic-coated spring steel wire and polymers. Flexible resilient reinforcing structures can be constructed of hydrophilic or at least partially hydrophilic materials such as sponge rubber, cellulosic sponge, hydrophobic polymer foams as well as foamed styrene butadiene, polyethylene, silicones and vinyl plastics.

Reinforced pads of the prior art are configured as elongated open frame structures defining at least one aperture and rounded ends. Shapes include a modified hourglass, a rectilinear, or a square outline formed by individual members defining at least one aperture which functions to provide the flexible resiliency necessary for retaining the absorbent pad in position after being deformed or twisted during wear. The open frame construction of reinforcing structures frequently position the most deformable portion of the structure and/or narrowest portion of the structure at a central position between the legs of the wearer so as to impart a bias against the legs of the wearer. This precludes the bunching and twisting of the pad and assists in the retention of the absorbent pad in position in the wearer's groin.

The manner in which a wearer fits or attaches an absorbent pad for use, however, is limited by the requirements for the extended wear and resulting shape of the structure of the absorbent pad to accommodate such extended wear. As noted above, absorbent pads with or without a framework generally have a construction of a rear portion, a central portion, and a forward portion. The central portion has a width that is narrower or equal to that of the forward and rear portions. Pads of the prior art require the removal of the wearer's outer clothing and undergarments, spreading of the legs, positioning of the absorbent pad in the groin, joining any adhesive portions or other retention mechanisms that are provided to hold the absorbent pad in position beneath the wearer's undergarments and then dressing.

Absorbent pads without frameworks require the wearer to manually ensure that the pad is properly spread open during and after positioning. An absorbent pad with a framework requires proper positioning of the deformable elements to retain the position of the absorbent pads between the legs of the wearer. As a practical matter, absorbent pads with and without frameworks are applied from the upper thigh region directly into the wearer's groin. The practical necessity for positioning the open absorbent pad in line with a longitudinal axis of the body ensures the proper positioning of the forward, central and rear portions of the absorbent pad. This method of application of the absorbent pad is required for proper positioning and the extended wear of the absorbent pad and make it impractical for positioning or applying under circumstances in which the wearer cannot disrobe.

It is therefore an object of the present invention to provide an apparatus and a method for using a urine-absorbing pad configured for rapid temporary positioning in the groin of a clothed wearer desiring to urinate and removal of the urine-absorbing pad from the groin of the wearer after urination that avoids the above limitations of the pads of the prior art.

Another object of the present invention to provide a urine-absorbing pad configured for rapid positioning in the groin of a clothed wearer desiring to urinate and removal of the urine-soaked pad without disrobing.

Yet another object of the present invention is to provide a urine-absorbing pad for emergency or medical use where a patient is immobilized or incapacitated and traditional forms of urine absorption are either not immediately available or would be impractical to use.

It is a further object of the present invention to provide a convenient and compact package containing one or more readily accessible urine-absorbing pads that permits a prompt response to the urgent need to urinate when no sanitary restroom facilities are available or during outdoor activities such as hiking, fishing, boating, golfing, or while traveling.

Still another object of the present invention is to provide a urine-absorbing pad configured for use with children in conjunction with toilet training.

### SUMMARY OF THE INVENTION

The above objects and other advantages are provided by the urine-absorbing pad assembly of the invention that is configured and constructed for use by a clothed wearer and which broadly comprehends an absorbing pad assembly that includes an absorbent member that is constructed to receive urine from a wearer and to distribute and retain the urine, and a longitudinal supporting member joined to, and supporting the absorbent member.

In one preferred embodiment, the absorbing member includes an outer layer or cover that contacts the wearer, an intermediate wicking layer that functions to distribute the urine and the absorbent material which absorbs and contains the urine.

The outer layer or cover of the pad assembly can be selected from natural and synthetic materials that have sufficient wet strength to resist tearing when the pad is inserted, but more importantly as it is removed from the wearer after use. This outer layer must be porous and highly permeable to the passage of liquid to the interior absorbent material. It is preferably formed from a hydrophobic material that will not retain moisture or remain wet after contact with the urine that is passed to the absorbing material below.

The intermediate wicking layer provides a porous, and preferably resilient flow path to distribute the urine from the point of discharge to the underlying absorbent material. It can be either hydrophilic or hydrophobic.

Any of the commercially available absorbent materials or media that have long been used in baby and adult diapers, incontinent pads and the like can be used in the absorbent layer of the pad assembly. The dry absorbent material can be used to fill a pouch or bag of highly porous material such as a lightweight non-woven or air-laid textile, a fine mesh, or the like. This sub-assembly can be used to facilitate mass production of the pad. As will be apparent to one or ordinary skill in the art, the construction of the absorbent member assembly can be varied by the addition and removal of layers or laminates and the substitution of other suitable materials, so long as the function and purpose of the absorbent member assembly is met.

As an alternative to the use of a separate subassembly containing the dry absorbent material, the outer covering can be used to retain the dry absorbent. In this construction, the outer material can form a portion of a bag or sack that surrounds the other sub-layers. In a preferred embodiment, a heat-sealable thermoplastic material over mesh construction can be used.

The supporting member has a smooth outer cover surface exhibiting a low coefficient of friction that is adapted for manually positioning the absorbent member in the groin of a wearer. The supporting member has sufficient compressive and tensile strength to maintain its structural integrity and to withstand the longitudinal pushing and pulling forces when the pad assembly is inserted for use and then removed for disposal.

The supporting member also exhibits sufficient flexibility along its longitudinal axis to assume the necessary curvature when positioned for use in the wearer's groin, with one end extending in contact with the lower abdomen at the end of the insertion step.

The supporting member can be joined to the absorbing member by a common outer cover that either partially or completely overlaps the outer surface of the supporting member, and the cover bonded, as by adhesive, heat sealing or other means known in the art. Alternatively, the supporting member can be joined by means of a pocket formed in the absorbing member for that purpose, in which case the supporting member can be removed after each use before the pad is discarded.

The longitudinal edges of the supporting member should not discomfort the wearer during use since the distal portion is positioned between the upper thighs and must pass in sliding relation against the typically sensitive skin area of that area. In one preferred embodiment the edges are provided with a border of a soft polymeric material having a low coefficient of friction. Most preferably, the border is a compressible material, such as foam, that can be compressed to form-fit the wearer and also to provide a dam or barrier to the liquid that is to be absorbed.

In a further preferred embodiment, the edges of the pad are formed with an elastic material that is stretched or extended during manufacture and joined to adjacent layers forming the pad. When relaxed, the elastic causes the pad to assume a slight curvature. This inherent curvature assists in positioning the pad and also in conforming it to the wearer's body during positioning and use.

The pad assembly has a proximal end which is gripped by the user between thumb and fingers for insertion of the distal end into the groin below the ureter in order to contain the urine until it is fully absorbed by the absorbent material in the pad. The distal end of the pad is preferably larger in surface area than the proximal end, which can be provided by a distal end of greater width, having an oval, elliptical or similar shape. Discharged urine will flow, under the effect of gravity, toward the distal portion, but will be absorbed and distributed by conduction and capillary action throughout the originally dry and compact absorbing material.

For reasons of economy and ease of mass manufacture, the supporting member can be formed from a single sheet or web of homogenous material or constructed from a plurality of components. Alternatively, the supporting member can be formed as a laminated structure and can incorporate even one or more components that function alone or in combination with the urine-absorbing member. It is preferably of a lightweight and made from non-allergenic materials. The supporting member can be provided with one or more longitudinal lines of perforations or foldlines, or it can be manufactured to have a transverse arcuate cross-section to facilitate fitting into the region between the wearer's upper thighs.

In yet another embodiment, the supporting member can be constructed from a highly resilient web or sheet having the desired characteristics of smoothness, a low coefficient of friction, a soft feel to the wearer and be impervious to urine, but which lacks the desired degree of tensile and compressive strength, and can be reinforced with one or more longitudinally-extending stays, ribs or struts to provide the necessary attributes to the completed supporting member.

In a further alternative embodiment, the ribs or struts are integrally formed, as by molding, of the same material as the rest of the supporting member. These ribs or struts can be visible on the exterior surface, or can be on the interior surface facing the absorbent portion of the pad assembly.

In a further preferred embodiment one or more of the integrally molded ribs or struts can also include a strengthening stay of a flexible material having the necessary compressive and tensile properties. One or more of these stays can project from the proximal end of the pad assembly to provide a gripping portion to facilitate its insertion and removal.

In yet another preferred embodiment, transverse ribs or struts provide the desired characteristics and shape for the transverse cross-section of the supporting member.

The structure and materials of construction of absorbing pad assembly are selected for ease of sliding insertion between adjacent clothing items and the wearer's body from the waist area to the groin region. Depending upon the nature of the wearer's clothes, the wearer may have to partially loosen or manually extend the waistband of one or more items of clothing.

Although the same general configuration for the pad assembly of the invention can be used by males and females, alternative constructions can be used for reasons of economy of manufacture and/or marketing purposes. For example, an absorption pad assembly for use by males can include a recess or pocket to better accommodate the male genitalia. In this embodiment, the supporting member is conveniently formed with the desired configuration and the absorbing material is likewise shaped for positioning in the pocket. With either a generally planar or the pocketed configuration, the construction will accommodate any swelling or expansion of the urine-absorbing medium following use of the pad so that it can be removed without loss of absorbed urine.

For one preferred form of packaging, the supporting member is sufficiently flexible and resilient to assume a rolled or coiled configuration for packaging in a tube and then unrolled and at least partially extended for insertion into position for use. As used herein, the term “resilient” means a supporting member that can be folded or rolled along its longitudinal and/or transverse axes and that will either rebound or that can be manually returned to substantially assume its original manufactured configuration.

In one preferred embodiment, a plastic wrapper, bag or other waterproof container is provided for receiving and disposal of the used pad. In a particularly preferred embodiment, the disposal bag is attached to the proximal end of the pad so that it can be rolled down over the pad as the pad is withdrawn from the wearer's clothing. The disposal bag or wrapper can also serve as the original protective packaging for the new pad assembly.

Among the advantages of the invention described herein is the ability of the absorbing pad to be discretely used under less than private conditions to provide urinary relief for a wearer. For example, a female wearer can discretely urinate using the absorbing pad in a standing or semi-squatting position with minimal need to loosen her clothing, thereby avoiding the use of an unsanitary restroom or other undesirable action when no restroom is available. Similarly, a male wearer can discretely insert the pad downwardly past the trouser waistband without opening the fly and urinate into the absorbing pad to avoid creating an unsanitary condition.

Although the absorbing pad assembly of the invention has been described with reference to its use as a urine-absorbing pad, it is to be understood that the absorbing pad assembly is suitable for the control of any type of fluid passed, discharged or exuded from the urinary or vaginal systems. The absorbing pad assembly can also be employed as a temporary dressing or bandage for the lower abdominal or groin region, in which case it can be maintained in a sterile condition prior to use by appropriate packaging and treatment, e.g., autoclaving, sterilizing radiation and other means known to the art.

### BRIEF DESCRIPTION ON THE DRAWINGS

The invention, together with its attendant advantages, will be further described below and with reference to the drawings in which the same and similar elements are referred to by the same number, and where:

FIG. 1 is a top side perspective view of one preferred embodiment of a urine-absorbing pad assembly constructed in accordance with the present disclosure;

FIG. 2 is a schematic cross-sectional view of the absorbing pad of FIG. 1 along line 2-2;

FIG. 3 is a top plan view of the pad assembly of FIG. 1;

FIG. 4 is a perspective view of one preferred embodiment of a supporting member of the absorbent pad of FIG. 1;

FIG. 5 is a perspective view of a second preferred embodiment of a supporting member for use with an absorbent pad of the invention having one or more structural elements and illustrating in phantom the longitudinal and transverse flexing of the supporting member;

FIG. 6 is a perspective view of another preferred embodiment of the pad assembly that is provided with a cup or recessed portion;

FIG. 7 is a cross-sectional side view of a supporting member for use in the pad assembly of FIG. 6;

FIG. 8 is a perspective view of another embodiment of an absorbing pad contained in a flexible packaging;

FIG. 9 illustrates clothed wearer inserting the urine-absorbing pad of the invention behind a waistband of the wearer's outer clothing and undergarment in preparation for use;

FIG. 10 is a side view of the urine-absorbing pad with a flexible impermeable wrapper or bag inverted and drawn to the proximal end and grasped in preparation for use;

FIG. 11 is a front perspective view of the urine-absorbing pad of FIG. 10 with the flexible impermeable disposal bag in place over the used pad in preparation for disposal;

FIG. 12 is a cross-sectional view illustrating another form of packaging for the pad;

FIG. 13 is a cross-sectional view of an alternative form of packaging for the absorbing pad of the invention; and

FIG. 14 is a side view, partly in phantom, of the pad assembly and disposal wrapper of FIG. 13 preparatory to sealing for disposal.

### DETAILED DESCRIPTION OF PREFERRED EMBODIMENTS

Referring now to the drawings in which like reference numerals identify the same or similar elements throughout the several views, and initially to FIG. 1, there is illustrated a first preferred embodiment of a urine-absorbing pad assembly 10, variously referred to herein as pad assembly, absorbing pad and pad. Absorbing pad 10 includes an absorbent member 20 and a supporting member 30 shown in phantom. Absorbing pad 10 has a distal portion 12, a proximal portion 14, an inner surface 16, an exterior surface 18 and an exterior edge or perimeter 19. The alignment of distal portion 12 and proximal portion 14 defines the central longitudinal axis of absorbing pad 10. In one preferred embodiment described in more detail below, the absorbent pad assembly 10 includes an upper moisture permeable layer or cover 21, an intermediate wicking layer 23 and an absorbent layer 25 that are carried by supporting member 30.

Absorbent member 20 is preferably formed from a plurality of layers or components that can be constructed as a subassembly and joined or fitted to supporting member 30. The absorbent material can be combined with nonwovens, plastic films and metal foils to form the composite structure or subassembly.

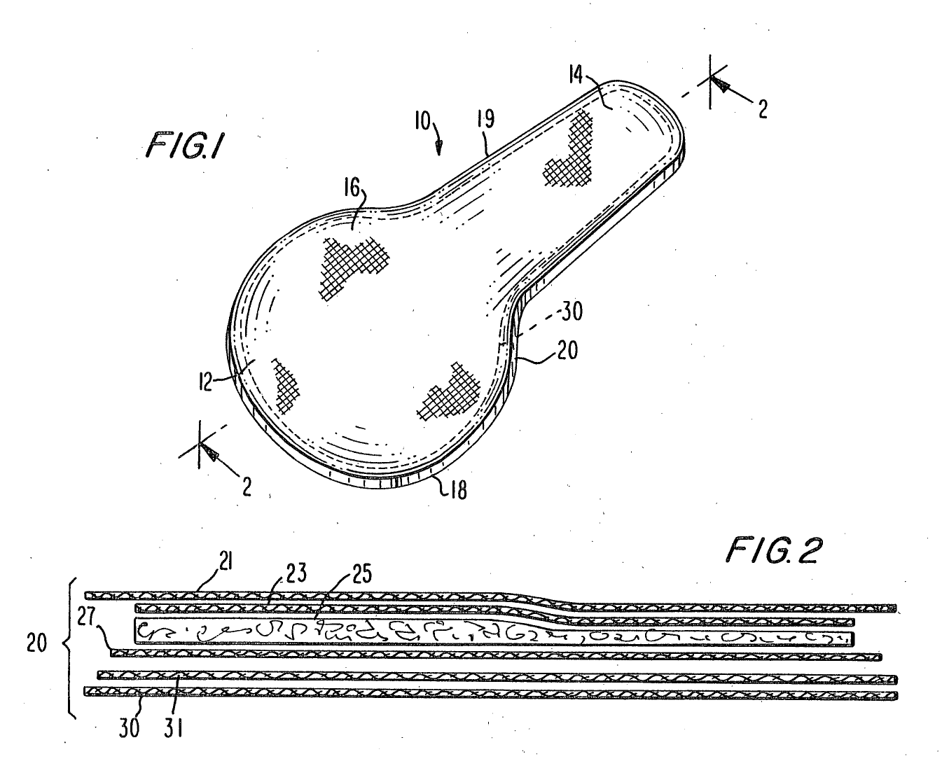

Referring to the cross-sectional view of FIG. 2, the absorbing member 20 is comprised of cover layer or layers 21, wicking layer 23 and absorbent layer 25. At the bottom is the supporting member 30. Also shown in cross-section are optional layers or elements, including a spine or supple metal web or foil that forms a supplemental supporting element or member 31 that provides additional support to withstand the compressive and tensile forces of insertion and withdrawal. An optional liquid-impervious layer 27 can be positioned below the layer of absorbent material 25.

As also shown in FIG. 2, the distal portion 12 of absorbent assembly 20 is thicker than the proximal end due to the greater quantity of absorbent material 25 provided to receive the urine that flows to the lower distal portion under the effect of gravity. In a preferred embodiment, the distal portion is at least about twice the thickness of the proximal end portion 14, and can be of triple the thickness.

As shown in FIG. 1, the pad assembly is preferably configured with a wider distal portion 12 and narrowed proximal portion 10. An overall length of about 13 inches, with a distal width of about five inches and proximal width of about two to three inches has been found to provide satisfactory capacity for adult use. The entire assembly can be made proportionally smaller for children or adults of less than average stature. Other shapes can also be employed based upon considerations of economy, ease of manufacturing and assembly, packaging and aesthetics, all of which will be apparent to those of ordinary skill in the field of personal care and sanitary products.

Moisture permeable cover layer 21 is formed from a soft and absorbent material that at least partially defines inner surface 16. Permeable layer 21 passes urine from the wearer into moisture wicking layer 23 and separates the wearer from the urine in wicking layer 23 and absorbent layer 25. Permeable layer 21 is hydrophobic and has a larger pore size than the sub-layers. Permeable layer 21 can be configured to expand in response to the liquid absorbed and the expansion of wicking layer 23 and moisture absorbent layer 25. Permeable layer 21 can be fabricated of materials in the form of a mesh web or a web of spun and/or air bonded synthetic polymeric filaments such as polyethylene, polypropylene and polyesters. Multiple layers 21 can be used to minimize contact by the wearer with the urine-soaked absorbent member following use.

Wicking layer 23 is positioned adjacent permeable layer 21 and disperses urine throughout its length and breadth to absorbent sub-layer 25. Wicking layer 23 can include conduits or passageways, to assist in distributing urine to absorbent layer 25. Wicking layer can be less hydrophilic than absorbent material 25 in order to distribute liquids across its surface, readily passing them through. Suitable materials for wicking layer 23 include non-woven fiber and filament webs of polypropylene, polyethylene and polyester.

Absorbent layer 25 is configured for the retention of urine and can have a uniform depth or thickness throughout, or a variable thickness with a maximum at the distal end 12 for the increased absorption of urine in that portion. Similarly, the overall amount of absorbing material positioned in absorbent layer 25 can vary depending upon its intended use.

Permeable layer 21, wicking layer 23 and absorbent layer 25 are described herein as separate layers, but it is understood the functions defined herein of these layers can be performed or combined into one or two layers of laminated or integrally formed materials having the characteristics required to perform these functions.

Suitable hydrophilic materials for absorbent layer 25 are superabsorbent dry flowable polymer materials used in infant diapers and can include acrylamides, fluffed absorbent type materials such as dry defibered cellulose and hydrophilic open cell polyurethane foams. Other absorbent materials and compositions presently known in the art or, developed in the future that have the capacity to absorb and retain the specified volume of liquid can be used in the pad assembly of the invention.

The preferred class of absorbent materials are the well-known and commercially available superabsorbent polymers that are available for use in personal care products. Superabsorbent polymeric materials include polyacrylate/polyacrylamide copolymers and sodium acrylate/acrylamide copolymers. Suitable products and their technical specifications are available from commercial suppliers, including Stockhausen Inc. of Greensboro, N.C. having an Internet address of www.stockhausen-inc.com/.

The manufacture and means of producing suitable absorbent materials are also well known from, and described in the patent literature. The disclosures of the following patents, as well as the references cited in these patents, are herein incorporated by reference: U.S. Pat. Nos. 6,623,848; 6,620,889; 6,565,981; 6,350,710; 5,408,019; and 4,605,401.

Optional impermeable layer 27 is at least co-extensive with, and adjacent to absorbent layer 25 and provides a barrier to contain any urine in permeable layer 21, wicking layer 23 and absorbent layer 25. In one preferred embodiment, impermeable layer 27 is a thin flexible web defining a liquid impermeable boundary layer and secures the exterior perimeter of absorbent member 20. Impermeable layer 27 can also be configured to have a smooth and/or soft surface which can include a flap or tab of material suitable for gripping as well as aiding unrestricted movement of absorbing pad 10 between the wearer and the wearer's clothes. Suitable materials for impermeable layer 27 include polymer or polyolefin films such as polyethylene or polypropylene.

Absorbent member 20 can also include an air gap or air space 26 positioned between any two of permeable layer 21, wicking layer 23 or absorbent layer 25. Air space 26 functions to absorb the fluid force of an intentional urination into layers 23 and 25 and thereby prevent the rapid introduction of urine into absorbent pad 20 from “spilling over” or “rolling off” onto the wearer. Air space 26, for example, can include segmented air encapsulations configured for collapse upon receiving urine.

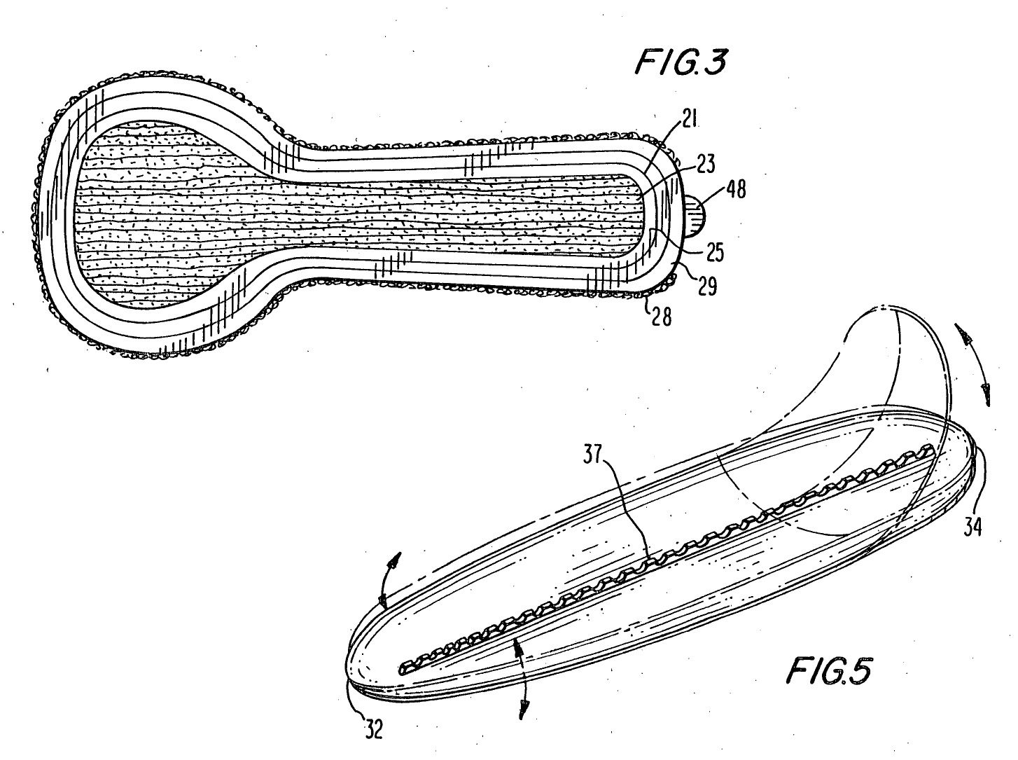

Referring now to the top plan view of FIG. 3, the relative positions of the cover layer 21 and wicking layer 23 are shown. In this embodiment, the cover 21 is heat-sealed to the optional impervious web 27 to create a subassembly containing the absorbent material 25. Also included is a cushioned resilient border 28 that can be formed from a fine closed cell polymer foam, a rolled edge of web material, or other means known to the art.

Absorbing pad 10 can also include an edge seal 29 positioned at, or proximate to perimeter 19. Edge seal 29 can be formed as a biased element suitable for adjustably sealing against the body of the wearer. Edge seal 29 can include, for example, arcuate or curled elements, resilient fluid filled elements, rubber, foamed or solid polymer materials that are known in the prior art and serve to provide a temporary seal against the wearer's body. Edge seal 29 can further include a removably covered adhesive wafer seal 48 for securely connecting perimeter 19 and preventing any loss of fluids from absorbing pad 10 after urination. The adhesive seal 48 of cushioned edge 29 can provide a temporary seal for use during packaging or a permanent adhesive connection to be applied only after use to facilitate disposal.

With further reference to FIG. 2, absorbing pad 10 is strengthened and stiffened in the direction of the longitudinal axis by optional supplemental support element 31 and manually grasped for the insertion and positioning in a wearer's groin for the absorption of urine passed by the wearer

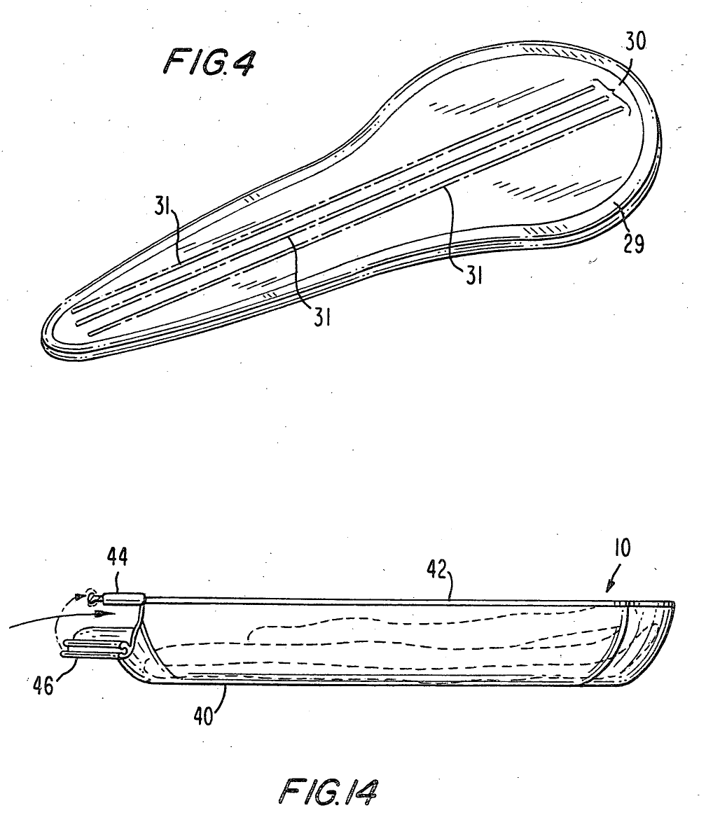

Referring now to FIGS. 4 and 5, there are shown alternative embodiments of a, supporting member 30 which provides support for the insertion and positioning of absorbing pad 10 in the groin of the wearer. Supporting member 30 includes distal portion 32 and a proximal portion 34 defining an elongated overall shape having a longitudinal axis, an inner surface 36 and an exterior surface 38.

Distal portion 32 preferably has an expanded elongate elliptical or oval shape having a rounded end 33 corresponding generally to the pad assembly shown in FIG. 1. The shape of distal end 33 is preferably arcuate and configured to support the posterior-directed insertion of absorbing pad 10 between the upper thighs and into the groin of the wearer. In one embodiment, distal portion 32 is configured to carry the weight of urine-laden pad 10 in conjunction with proximal portion 34 during use and removal after use by the wearer. Distal portion 32 preferably has a width equal to or greater than the width of proximal portion 34. It should be understood that the thickness and shape of distal portion 32 can vary depending upon its intended use, including factors such as the desired maximum amount of urine to be retained, whether the use is by an adult or a child and the overall body type of the user.

With further reference to FIG. 4, supporting member 30 can have one or more elongated elements 31 that are interconnected or separately positioned in absorbent member 20. The inclusion of multiple reinforcing elements 31 in supporting member 30 serves to maintain the longitudinal stiffness, transverse flexibility, alone and in combination as previously described. As also illustrated by the broken lines 39 in FIG. 4, the supporting member 30 can be provided with foldlines to facilitate the transverse curving of the pad 10.

REFERRING to FIG. 5, an alternative configuration of supporting member 30 is fabricated from non-corrugated paperboard or a lightweight solid plastic or a foamed and compressed polymer, such as foamed polystyrene, that is stiffened in the direction of its longitudinal axis and can additionally include one or more longitudinally-aligned axial stiffening members. As illustrated, the axial stiffening member 37 is notched to facilitate bending along the longitudinal axis, as shown in phantom, and still retain the required degree of longitudinal stiffness and/or resistance to compression. Alternatively, axially stiffening members can define a preformed longitudinal channel having an arcuate or “V” shaped transverse cross-section that can be selectively flexed also as shown in phantom, or assume a flat or planar configuration for bending about a transverse axis (not shown).

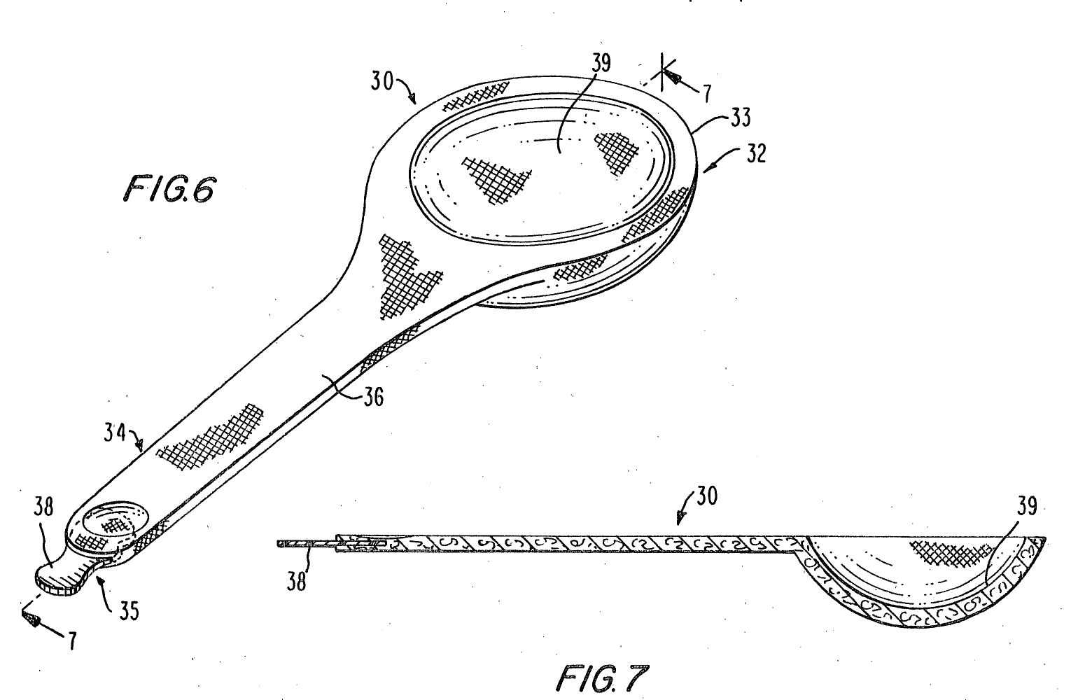

With reference to FIGS. 6 and 7, distal portion 32 includes a recess or cupped portion 39 for accommodating an increased volume of absorbent member 20 and/or to facilitate in configuring distal end 12 as a flexible and bendable portion of pad 10. When distal portion 32 includes recessed area 39, absorbent member 20 is configured to fill the recess with an increased volume of absorbent member 20. The configuration of FIG. 6 can also be used to accommodate use of the pad assembly by a male, as will be described in more detail below.

Proximal portion 34 is configured to assist the wearer in inserting absorbing pad 10 into position in a wearer's groin and its terminal end 35 can also include a grip 38 suitable for gripping such as a flexible tab, finger loop, small handle or finger grip. Proximal portion 34 preferably has a tapered width increasing towards the distal portion 32. The proximal portion 34 is constructed with sufficient rigidity, tensile and compressive strength to support the pad assembly during the posterior directed insertion into position for use and its anterior directed removal after use.

Supporting member 30 preferably has a deformable and flexible structure having suitable stiffness along the longitudinal axis so that at least the distal portion can be inserted into the groin of the wearer in a posterior direction. The assembly also has sufficient structural integrity transversely to retain its shape to permit the absorption and retention of liquids by absorbent member 20. Supporting member 30 is preferably formed from an impermeable material.

In order to accommodate a male wearer, recess 39 in distal portion 32 is extended to include an increased volume of absorbent member 20 and comfortably accommodate male genitalia. Alternatively, when distal portion 32 defines a hole 39, absorbent member 20 can be configured with a cupped shape to accommodate an increased volume of absorbent member 20 and/or male genitalia. In this embodiment, proximal portion 14 can be minimized.

Structure 30 has sufficient strength along the longitudinal axis to permit inserting of absorbing pad 10 between the thighs and into the groin region of a wearer in a generally posterior direction without collapsing or being permanently deformed. Structure 30 can be a rigid member or a flexible member, but preferably exhibits flexibility and resilience along the transverse axes, and has sufficient transverse flexibility to maintain a width suitable for receiving and retaining urine from the wearer.

The flexibility of the pad assembly enables selected portions of supporting member 30, such as distal portion 32 to be longitudinally stiff for insertion and simultaneously to accommodate the transverse flexing or bending of proximal portion 34 to align with the shape of the natural curvature of the groin. In other applications, such as those configured for male use, distal portion 32 in conjunction with proximal portion 34, can form a pocket or recess for receiving urine.

Materials of construction for supporting member 30 can include a polymer-coated or other cellulose material, paperboard, expanded and compressed polystyrene foam sheets, as well as flexible or deformable polymers, thin metals or metal foils, composites and combinations thereof. Supporting member 30 can be fabricated from one or more plastic-coated paperboard strips and otherwise reinforced in accordance with methods known to the art to provide additional rigidity. Member 30 can be formed by die-cutting from webs, injection or compression molded, or by other methods well known in the art for producing relatively thin flexible articles.

Supporting member 30 can be integrally formed with impermeable layer 27, integrally connected to any layer in absorbent member 20, or be removably attached to form an assembly with absorbent member 20.

Referring now to FIG. 8, an alternative configuration is illustrated where the distal and proximal ends are of approximately the same width. As previously noted, absorbing pad 10 is preferably a disposable, single use device for the containment of urine from a wearer, but absorbing pad 10 can also be configured to contain multiple urinations. For example, wearers experiencing the condition known as frequent urination typically pass small amounts of urine, but have the urge to urinate more frequently than the average person, and finding or staying close to a restroom may be very inconvenient. Absorbing pad 10 can be positioned when there is a need to urinate and either left in place, or, removed after urination, securely contained and then placed into the groin of the wearer again when there is an additional need to urinate. In this embodiment, absorbing pad 10 can include a thicker permeable layer 21 to provide additional separation between the wearer and wicking layer 23 and absorbent layer 25. Absorbent layer 25 can also include a greater volume of absorbent material. The proximal portion can also accommodate more absorbent material and therefore can contain more liquid.

The embodiment of FIG. 8 can also be used for post-surgical patients or by those suffering from bladder control problems, where small amounts of urine or other fluids are discharged over an extended period of time. The wearer sensing the eventual accumulation, will know when to remove the pad.

In the embodiment shown in FIG. 8, distal portion 12 and proximal portion 14 have a uniform width. Supporting member 30 in this embodiment is both flexible and stiff. For a selected distal portion, member 30 can be longitudinally rigid for insertion into the groin, while the proximal portion can be flexed in region 37 about the lower abdomen. Region 37 can be provided with preformed angular V-bend or have an arcuate cross-section transverse to the longitudinal axis. Alternatively, flexible region 37 can include one or more spaced-apart fold lines or weakened areas to facilitate flexing or bending transversely, while retaining the required degree of longitudinal stiffness. Distal portion 32 can retain its planar configuration or flex along the longitudinal axis to enhance stiffness, while simultaneously, proximal portion 34 is flexed transversely. This combination of bending facilitates insertion into the posterior groin region of the wearer while clothed.

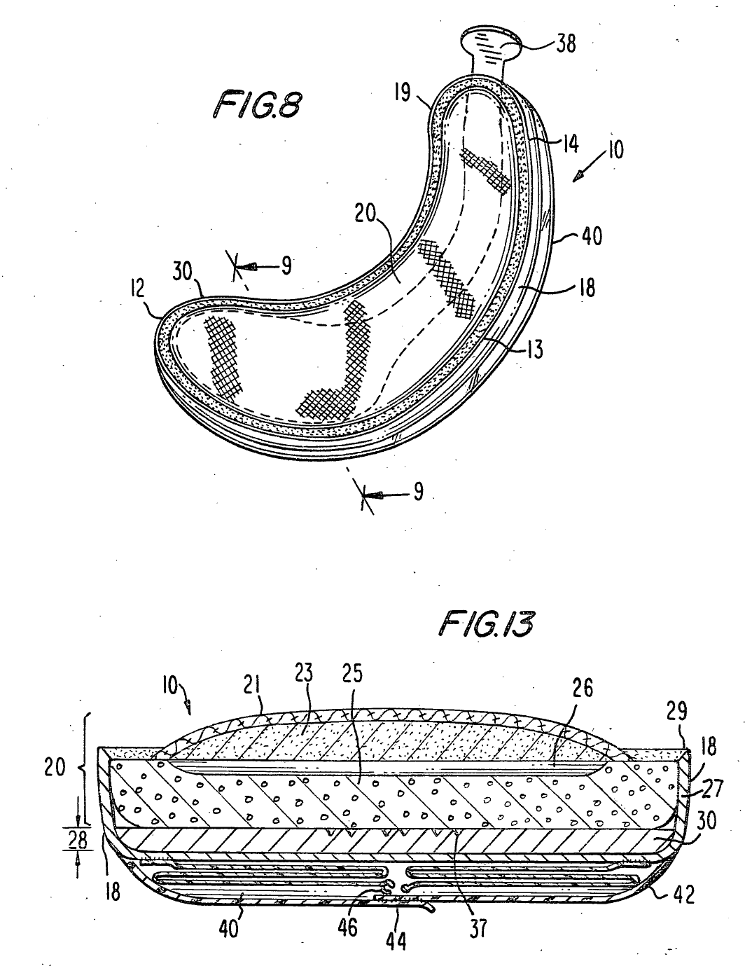

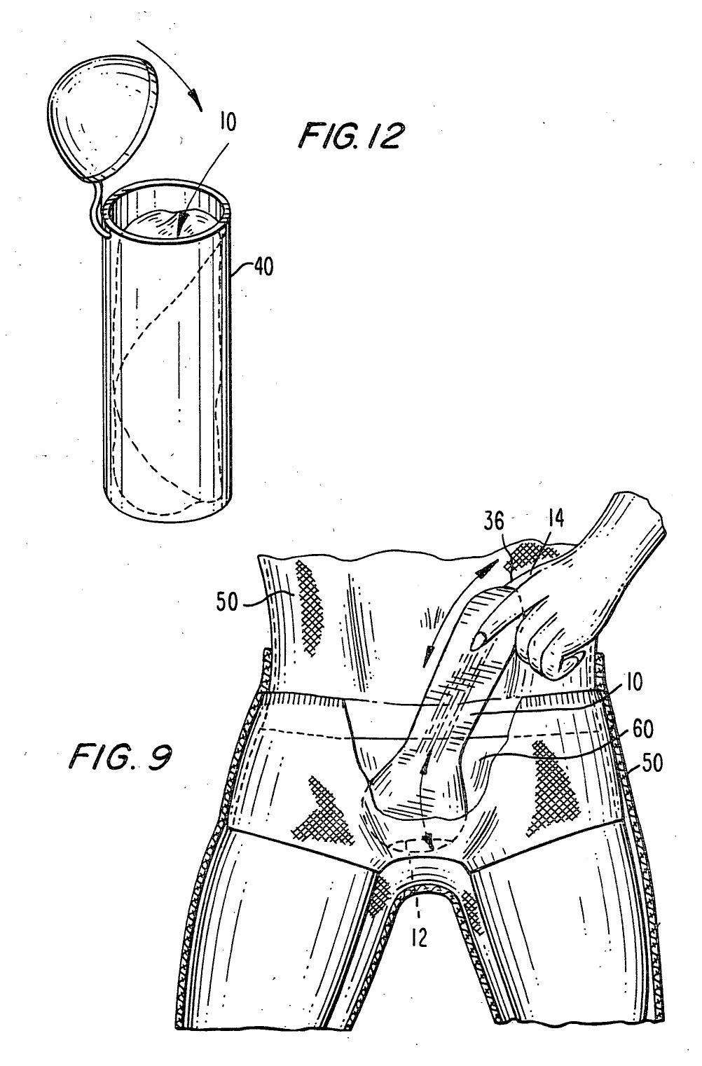

Referring now to FIG. 9, which illustrates the method of using of the pad, the wearer grips proximal portion 14, inserts the distal portion 12 generally aligned with the longitudinal axis of absorbing pad 10 along the abdomen and then in the posterior direction into the groin and between the thighs of the wearer. The posterior-directed movement of absorbing pad 10 is continued until distal end 12 is positioned for use by the wearer. Absorbing pad 10 is configured to extend from the region of the groin and/or lower abdomen to encompass the area in proximity to the urethric orifice, and below and to the rear. When properly positioned for use, the pad receives and contains discharged urine until it is distributed and absorbed by the absorbent material. Depending upon the intended use of absorbing pad 10, both the distal portion 12 and proximal portion 14 can be constructed to receive and absorb urine from the wearer.

As will be understood from FIG. 9, the pad as illustrated and described is designed for use when the wearer is standing, seated or partially squatting. Discharged urine will flow under the effect of gravity to the distal portion of the pad where it is contained until absorbed by the superabsorbent material.

For female applications, supporting member 30 has the structural integrity to support the manual positioning of absorbing pad 10 by the grasping of proximal end 34 and inserting distal end 32 in a posterior direction into the groin region or between the junction of the thighs and the lower abdomen. This inserting of absorbing pad 10 positions proximal portion 14 above the groin and/or proximate the lower abdomen and distal portion 12 in the region of the female urethric orifice for the absorption of urine. Distal end 12 preferably extends in the posterior direction beyond the proximity of the ureteric orifice and/or the region necessary for receiving and containing urine from the female to contain any urine that cannot be absorbed until it is wicked into the adjacent absorbent material. The female can remain fully clothed and may be standing, squatting or partially seated when absorbing pad 10 is inserted into position for use.

In preparation for use, the wearer removes any packaging or outer wrapper. When supporting member 30 is a removable component of absorbing pad assembly 10, preparation for use includes the installation of supporting member 30 into the sleeve or pocket 28 provided in absorbent member 20. The handle 36 or tab 38 of support member proximal portion 14 assists in the positioning of absorbing pad 10.

Supporting member 30 alone, or with absorbing member 20 are grasped by proximal portion 14 and distal portion 12 is manually inserted between clothing 50 of the wearer and the wearer's body 60 from the waist area, for example, by pulling in the abdomen and/or extending the clothing's waistband, or both. Depending upon the nature of the clothing and body type, a wearer may have to partially loosen one or more portions of outer clothing. Inner surface 16 and outer surface 18 of absorbing pad 10 are smooth and exhibit low friction resistance to facilitate the manual insertion along the wearer's abdomen and then in the posterior direction into the groin while in sliding contact with the wearer's clothes 50.

As required, a wearer can preliminarily deform or bend portions of absorbing pad 10 into a suitable shape, such as a “V” shape, before or during its insertion to provide an enhanced seal against the wearer's body 60. Distal end 12 is inserted into the groin proximate the buttocks of the wearer and may be curled or bent along the longitudinal axis, as required, to selectively enhance or facilitate insertion and/or final positioning. This shaping can be accomplished by manually pressing on the pad assembly through the wearer's clothing with the free hand while advancing the pad.

Wrapper 40 can be integrally connected to pad 10 and left in position on outer surface 18, or opened during use. When wrapper 40 is opened, pad 10 is grasped through the wrapper so that it extends up and around the wearer's fingers as shown in FIG. 10.

Once positioned for use, the wearer urinates into absorbing pad 10. Absorbing pad 10 is held in position during urination by the bias inherent in absorbent member 20 and/or member 30, manually by the wearer, and/or by the wearer's clothes. Supporting member 30, when configured as a removable element, can be removed at any time after the absorbent member is positioned for use. The removable supporting member 30 can be disposable, but is preferably reusable for reasons of economy and environmental interests.

For use by a male, pad 10 is manipulated for positioning over the male genitalia and held in position during use. Pad 10 in this embodiment is especially configured to absorb and minimize deflecting the discharge during use. In this embodiment, an enlarged handle 36 or tab 38 can be provided to assist in the positioning or removal of pad 10.

After urinating into absorbing pad 10, the wearer grasps proximal portion 14 and withdraws absorbing pad 10 from between the wearer's clothes 50 and body. The wearer can also slide wrapper 40 over pad 10 during the withdrawal as shown in FIGS. 10 and 11. Absorbing pad 10 can be at least partially folded or securely placed within wrapper container 40 before or after being withdrawn from the wearer, and then discarded when convenient.

With reference to FIG. 11, container 40 is shown extended from its originally rolled or folded position on the proximal end of pad 10. In the preferred embodiment illustrated, container 40 is a sleeve or wrapper of thin, flexible tear-resistant moisture impermeable material having an opening 42 to receive absorbing pad 10 and closing means 43 that preferably provides a fluid-tight seal and can include a continuous pressure snap seal as illustrated, an adhesive seal, tie-off strips or a drawstring.

In the first position, container 40 is conformingly folded or positioned on outer surface 18 or support 30. Container 40 can be a portion of impermeable layer 27 or a separate layer. In the enclosed position, container 40 has been unfolded, inverted around absorbing pad 10, and opening 42 defined, and closing means 44 secured. When supporting member 30 is removable supporting member 30 can be enclosed in container 40 or, alternatively, be configured for storage as a stand-alone item.

Container 40 in the storage position can function as a sanitary seal around absorbing pad 10 prior to and after use. In this configuration, container 40 is opened, absorbing pad 10 is repositioned from a stored position to a position suitable for use, container 40 is either detached or placed into a position generally conforming with exterior surface 18, and container 40 is then inverted and sealed around absorbing pad following use.

Referring now to FIG. 12, a container for the assembly 10 can take the form of a lightweight cylindrical canister-fitted with a cap or removable end cover, made from paperboard or thin-walled plastic box suitable for storage before and after the use of absorbing pad 10. The dimensions of the cylindrical canister of FIG. 12 are suitable to allow for the arcuate bending or flexing of structure 30 into, for example, a rolled configuration, and retain the required longitudinal stiffness of structure 30. The configuration of the container can be selected for convenient storage in a woman's purse, automobile glove compartment or pocket in a pair of pants, jacket, or backpack. For example, the separate absorbing pads 10 stored in canisters of FIG. 12 can be connected by external plastic connectors similar to those used with beverage six-packs.

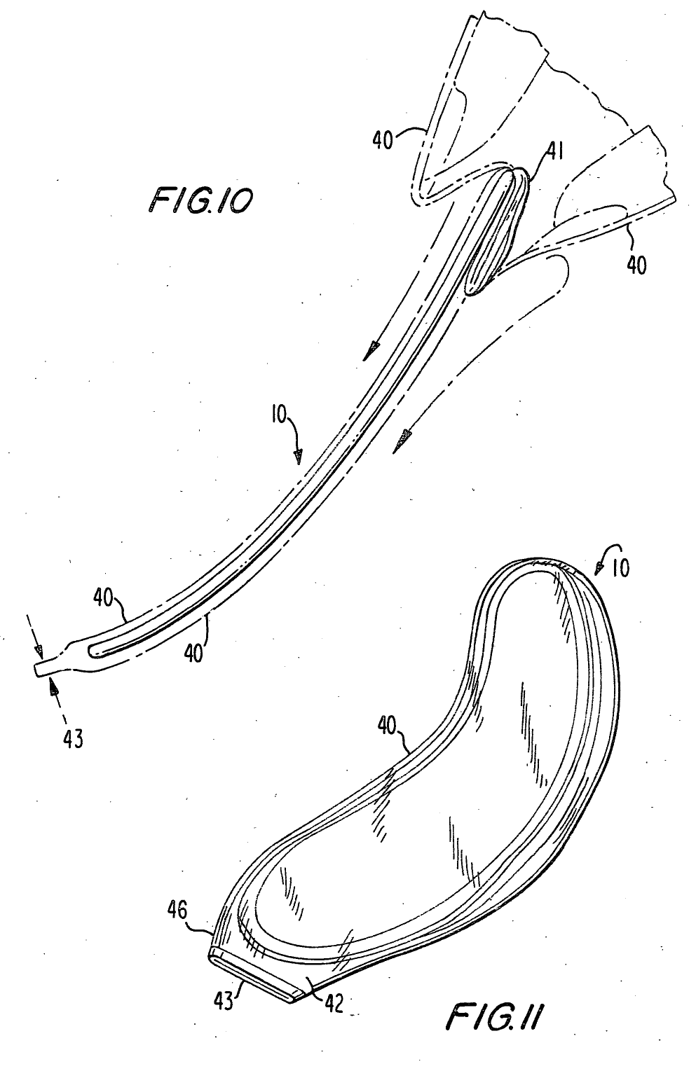

As further shown in FIGS. 13 and 14, an optional packaging container 42 is positioned on the lower or exterior portion of assembly 10. In the embodiment illustrated, the packaging 42 has a longitudinally extending closure 44 in the form of a pull tab or releasable adhesive. When opened following use, access is provided to a flexible wrapper 40 that is shown compactly folded, but which when opened and extended can be wrapped around the wet pad and sealed along interlocking air-tight edge seal 46 pending disposal, as best shown in FIG. 14. Other methods and constructions for packaging, wrapping and sealing the used pad will be apparent to those of ordinary skill in the art.

In an alternative preferred embodiment illustrated in FIG. 13 supporting member 30 serves as a reusable applicator for the positioning of the absorbent member 20 and is then withdrawn from the absorbing pad assembly 10 after positioning the absorbing member 20 for use. In this embodiment, absorbent member 20 is provided with a pocket or sleeve 28, shown in FIG. 13, for receiving supporting member 30 and permitting its easy removal. As shown in FIGS. 6 and 8, member 30 can be provided with a projecting and enlarged gripping portion 38 at its proximal end. Absorbent member 20 is constructed with sufficient wet-strength to be manually withdrawn following use.

As shown in FIG. 13, supporting member 30 is provided with longitudinal grooves 37 to facilitate transverse flexing during fitting of the pad assembly 10 for use. An impermeable barrier layer 18 of a heat sealable polymeric material surrounds the edges and bottom of the support 30 and can be heat sealed or otherwise bonded to cover layer 21 to provide a smooth, snag-free surface.

As will also be understood from FIGS. 13 and 14, absorbing pad 10 lends itself to stacking or other positioning of multiple units in a conveniently-sized storage container or package. Before use, the pad can be compressed and vacuum sealed in a sanitary, sterilizable wrapper to minimize storage space requirements.

The intended use of the absorbing pad of the invention also lends itself to sale from vending machines, particularly in highway service stations, marinas, parks and other locations where sanitary restroom facilities are not conveniently available. Packaging having the exterior configuration of a 12-ounce beverage container can be stocked in vending machines and sold at the same price as the beverages.

The inherently novel structure and the ability to insert the pad between the wearer's body and clothes and into position without disrobing can advantageously provide relief to a broad range of wearers when restroom facilities are unavailable and the wearer is in less than private surroundings, such as those of an automobile or a relatively secluded area.

The absorbing pad of the invention can also be used by those who are wheelchair-bound or who are otherwise capable of only limited mobility, e.g., arthritis sufferers. Use of the pad by such individuals eliminates the often severe burden on the individual and the caregiver(s) in dealing with movement from a wheelchair to a toilet and back. The ability to insert the pad relatively quickly and easily eliminates the undesirable circumstance of the wearer remaining wet in a conventional incontinent pad or adult diaper until a caregiver can make the change.

The pad of the invention also provides a handy and discreet device for use by a victim of a sexual assault to collect and retain fluids to be used in DNA analysis. Pads can be supplied to police and EMS personnel in storage containers and provided to the victim in a sterile condition to satisfy evidentiary requirements of any later legal proceedings.

The absorbent pad of the invention can also be advantageously used by emergency medical service personnel with unconscious or incapacitated personnel in order to absorb the uncontrolled release of urine following an accident or admission to the emergency room. The pad can quickly and discreetly be placed in position by medical technicians without exposing the wearer or taking time to at least partially undress the wearer. Its use will also avoid the possible embarrassment and resistance of a conscious patient who is under treatment who must urinate, but cannot be provided with appropriate facilities, e.g., in an EMS vehicle. Use under such circumstances can avoid an unsanitary condition in an ambulance or emergency room treatment area and the attendant additional clean-up time, delay and expense.

While the use of absorbing pad 10 has been described herein in conjunction with an at least substantially clothed or encumbered wearer, it is understood that absorbing pad 10 can be positioned for use without any external clothing, sheets or medical dressings.

Although the illustrative embodiments of the present disclosure have been described herein with reference to the accompanying drawings, it is to be understood that the disclosure is not limited to these specific embodiments. Changes and modifications will be apparent to one of ordinary skill in the art without departing from the scope or spirit of the invention which is to be determined with reference to the following claims.

We claim:

1. A urine-absorbing pad assembly for receiving urine discharged by a wearer comprising:
an elongated liquid-absorbent member having a distal portion configured to pass between the wearer's thighs in close-fitting relation to the wearer's body and to receive urine discharged by the wearer and a proximal portion to contact the wearer's lower abdominal region; and
a longitudinal supporting member joined to, and extending between the proximal and distal portions of the absorbent member,
the supporting member being longitudinally inextensible and incompressible,
the proximal portion of the supporting member extending to permit the gripping of the supporting member between a thumb and at least one finger,
whereby the pad assembly can be manually inserted between the wearer's body and adjacent garments to position the pad assembly to receive the discharge of urine and to thereafter removed and discarded.

2. The absorbing pad assembly of claim 1, wherein the supporting member is permanently joined to the absorbent member.

3. The absorbing pad assembly of claim 1, wherein the distal end of the supporting member includes a cup-like recessed portion.

4. The absorbing pad assembly of claim 1, wherein the length of the supporting member and the absorbent member are sufficient to extend from the lower abdomen to a posterior position beyond the ureteric orifice of an adult user.

5. The absorbing pad assembly of claim 1 in which the liquid absorbent member comprises a moisture permeable outer layer, an intermediate wicking layer and an absorbent layer.

6. The absorbing pad assembly of claim 5, wherein the absorbent member further includes a moisture impermeable barrier layer below the absorbent layer.

7. The absorbing pad assembly of claim 1, wherein the supporting member is separable from the absorbent member.

8. The absorbing pad assembly of claim 7, wherein the absorbing member includes a sleeve for receiving the support member that is positioned below the absorbent material.

9. The absorbing pad assembly of claim 1, wherein the supporting member is flexible along its longitudinal axis.

10. The absorbing pad assembly of claim 1 which further comprises a flexible container for at least receiving the absorbent member that is attached to a portion of the assembly.

11. A urine-absorbing pad assembly that can be used by a wearer without disrobing comprising:
an elongated liquid-absorbent member having a distal portion configured to pass between the wearer's thighs and be positioned in close-fitting relation to the wearer's body and a proximal portion that contacts the wearer's body, the distal portion and the proximal portion being configured to receive urine discharged by the wearer, the absorbent member including a moisture permeable, smooth outer layer that contacts the wearer's body; and
a longitudinal supporting member joined to, and extending between the proximal and distal portions of the absorbent member and defining a longitudinal axis,
the supporting member being flexible and resistant to compression and extension along its longitudinal axis;
the proximal portions of the supporting member including an end portion to be manually gripped between a thumb and at least one finger, to thereby permit the pad assembly to be manually inserted between the wearer's body and adjacent garments without disrobing to position the pad assembly to receive a discharge of urine and to thereafter be removed for disposal.

12. The absorbing pad assembly of claim 11, wherein the absorbent member and supporting member are transversely flexible along their respective longitudinal axes to conform to the wearer's body.

13. The absorbing pad assembly of claim 11, wherein the permeable outer layer is formed from a synthetic material exhibiting a low co-efficient of friction with the wearer's skin.

14. The absorbing pad assembly of claim 11 which further comprising at least one impermeable barrier layer positioned below the absorbent material and constituting a portion of either the absorbent member, the supporting member, or both.

15. The absorbing pad assembly of claim 14, wherein the barrier layer forms a pocket for receiving the supporting member.

16. The absorbing pad assembly of claim 11, wherein at least a portion of the absorbent member includes an edge seal.

17. The absorbing pad assembly of claim 11, wherein at least one of the supporting member and absorbent member include a flexible fluid impermeable container that is extendable to enclose the pad assembly.

18. The absorbing pad assembly of claim 1, wherein the absorbing pad further includes a container for receiving the absorbing pad

19. The absorbing pad assembly of claim 18, wherein the container is formed from a layer of impermeable material having an opening for receiving the absorbing pad and includes a closure.

20. The absorbing pad assembly of claim 19, wherein the closure provides a fluid-tight seal for the container.

21. A method of containing and disposing of urine discharged by a clothed human subject comprising:
providing an elongated urine-absorbing pad assembly that includes a liquid-absorbent component, the pad assembly having a distal portion and a proximal portion;
manually gripping the proximal portion of the pad assembly;
inserting first the distal portion of the urine-absorbing pad assembly behind the waistband of any clothing worn by the subject,
passing the pad assembly between the subject's body and clothing in a generally posterior direction to position the pad assembly between the legs and in the groin region of the subject for the absorption of urine discharged by the subject; and
removing the urine-absorbing pad from contact with the subject after urination for disposal.

22. The method of claim 21, wherein the subject is standing or partially-squatting during the positioning of the pad between the subject's body and clothing.

23. The method of claim 21, wherein the distal portion of the urine-absorbing pad is closely fitted into contact with the subject's groin.

24. The method of claim 21 which includes manually maintaining the urine-absorbing pad in position during urination.

25. The method of claim 21 in which the urine-absorbing pad assembly further includes a supporting member and the method further comprises removing the supporting member from the pad assembly prior to disposal.

26. The method of claim 25 in which the supporting member is generally co-extensive with the pad assembly, and the method includes manually gripping a proximal end of the supporting member and guiding the pad assembly into position for use.

27. The method of claim 21 which further includes placing the used pad assembly in a flexible container for disposal after removal from contact with the subject.
